(19) United States
(12) Patent Application Publication
(10) Pub. No.: US 2026/0118300 A1
(43) Pub. Date: Apr. 30, 2026

(54) DETECTION DEVICE

(71) Applicant: Kabushiki Kaisha Toshiba, Tokyo (JP)

(72) Inventors: Akira FUJIMOTO, Kawasaki Kanagawa (JP); Yosuke AKIMOTO, Yokohama Kanagawa (JP); Yoshihiko KURUI, Chigasaki Kanagawa (JP)

(21) Appl. No.: 19/272,648

(22) Filed: Jul. 17, 2025

(30) Foreign Application Priority Data
Oct. 24, 2024 (JP) 2024-187115

(51) Int. Cl. G01N 25/18; G01N 33/00

(52) U.S. Cl. CPC G01N 25/18; G01N 33/0027

(57) ABSTRACT

According to one embodiment, a detection device of an embodiment includes a package having a lid, and a sensor substrate that is located inside the package and holds a sensor chip. The lid has two or more holes in a second region which is a region outside a first region which is a region facing the sensor chip in the lid.

### CROSS-REFERENCE TO RELATED APPLICATION(S)

This application is based upon and claims the benefit of priority from Japanese Patent Application No. 2024-187115, filed Oct. 24, 2024, the entire contents of which are incorporated herein by reference.

### FIELD

Embodiments of the present invention relate to a detection device.

### BACKGROUND

The detection device can measure the concentration of gas in a package in which the sensor chip is disposed. At this time, when the gas concentration is measured, it is necessary to correct for variations in temperature, humidity, and atmospheric pressure. When a transient response of the concentration of the gas occurs in the package, it is necessary to equalize the response speeds of the sensors in order to realize accurate correction in the transient response. The response speed of each sensor depends on the flow velocity of the gas flowing in the package in which the sensor is disposed.

### BRIEF DESCRIPTION OF THE DRAWINGS

FIG. 1A is a perspective view and FIG. 1B is a schematic sectional view of a detection device 1 according to a first embodiment.

FIG. 2 is a schematic sectional view showing a part of the detection device 1 according to the first embodiment.

FIG. 3A-3C are schematic views showing an example of the periphery of a sensor substrate.

FIG. 4 is a schematic view showing an example of the lid 18 in the first embodiment.

FIG. 5 is a simulation result of a flow of gas in the vicinity of the detection device 1.

FIG. 6 shows the results of simulation of the velocity of the gas flow with the position of the hole 20 changed.

FIG. 7 is a schematic view showing an example of the lid 18 in the first embodiment.

FIG. 8 shows the results of simulation of the velocity of the gas flow with the hole diameter changed.

FIG. 9 is an example of a simulation result of a flow of gas in the vicinity of the detection device 1.

FIG. 10 is a graph showing the velocity distribution measured along the X-axis.

FIG. 11A-11B are schematic views showing an example of the lid 18 in the first embodiment.

FIG. 12A-12B are schematic views showing an example of the lid 18 in the first embodiment (first modification).

FIG. 13 is a schematic sectional view showing a part of the detection device 1 according to the second embodiment.

FIG. 14A-14B show the results of simulation with different hole diameters.

FIG. 15A-15B show the results of simulation with different hole diameters.

FIG. 16A-16B show the results of simulation with different distances h2 between the lid 18 and the sensors.

FIG. 17 is a schematic view showing the relationship between the gas flow and the diffusion direction.

FIG. 18 is a graph showing a change in normalized diffusion amount N_{z,t}/N₀ with time.

### DETAILED DESCRIPTION

In general, according to one embodiment, a detection device includes a package having a lid, and a sensor substrate positioned inside the package and holding a sensor chip. The lid has two or more holes in a second region which is a region outside a first region which is a region facing the sensor chip in the lid.

Hereinafter, embodiments will be described with reference to the accompanying drawings. In the embodiments, substantially the same components are denoted by the same reference numerals, and the description thereof may be partially omitted. The drawings are schematic, and the relationship between the thickness and the planar dimension of each portion, the ratio of the thickness of each portion, and the like may be different from actual ones.

### First Embodiment

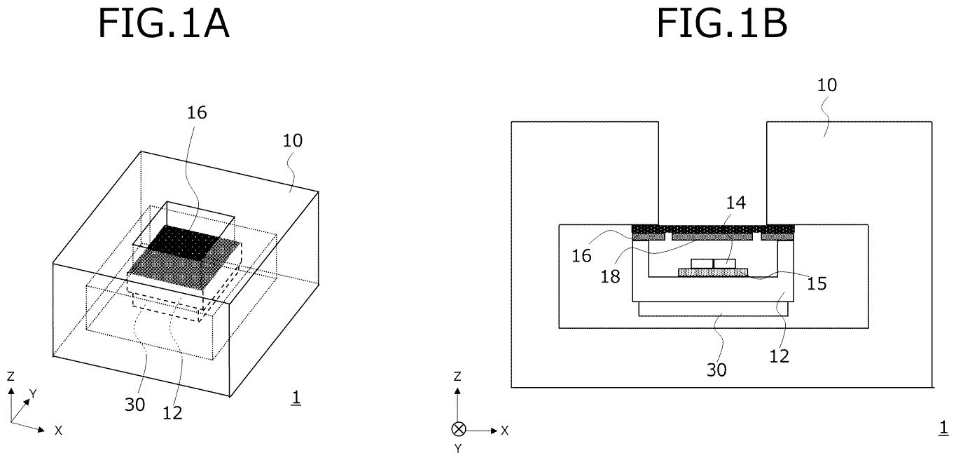

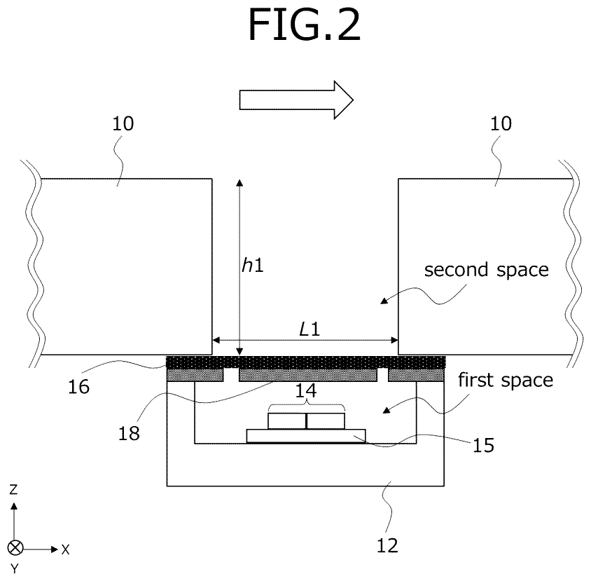

FIG. 1A is a perspective view of a detection device 1 according to the present embodiment, and FIG. 1B is a schematic cross-sectional view thereof. The detection device 1 has a housing wall 10, a package 12 is arranged in a space surrounded by the housing wall 10, and a sensor substrate 15 having a sensor chip 14 is arranged in the package 12. A battery 30 for supplying power to the sensor substrate 15 is provided below the package 12. The space outside the housing wall 10 and the space inside the package 12 are separated by the membrane 16 and the lid 18. The shape of the detection device 1 shown in the drawings is merely an example, and for example, the shape of the housing wall 10, the position, size, shape, and the like of the package 12 are not limited to the aspects shown in the drawings. FIG. 2 is a schematic cross-sectional view showing a part of the detection device 1. The detection device 1 according to the embodiment includes a housing wall 10, a package 12, a sensor chip 14, a sensor substrate 15 that holds the sensor chip 14, a membrane 16, and a lid 18. The package 12 has an opening on the upper surface, and the opening is closed by a flat lid 18. A membrane 16 is disposed on the opposite side of the package 12 from the lid 18. In the example shown in the figures, the membrane 16 is applied over the lid 18, so that the lid 18 and the membrane 16 are in contact. The vertical relationship between the membrane 16 and the lid 18 may be as shown in the figure, or the membrane 16 may be disposed between the lid 18 and the package 12. According to the present embodiment, there is a space partitioned from the outside by the lid 18, the membrane 16, and the package 12. This space will be hereinafter described as a “first space”.

On the other hand, a space surrounded by the housing wall 10 and the membrane 16 is referred to as a “second space”. The bottom surface of the second space is the membrane 16, and the side surface of the second space is the housing wall 10. The surface facing the bottom surface of the second space is an opening of the housing wall 10, and thus the second space is an open system. In the present embodiment, when the depth of the second space, that is, the dimension perpendicular to the membrane 16 at the bottom, is denoted as h1, if the dimension h1 is significantly smaller than the distance L1 between the housing walls 10, no vortex is generated in the second space. Conversely, if the dimension h1 is significantly larger than the distance L1, multiple turbulences occur in the second space, and the flow velocity at the bottom of the second space decreases. Therefore, it is preferable that h1 is between ½ and 3 times the distance L1.

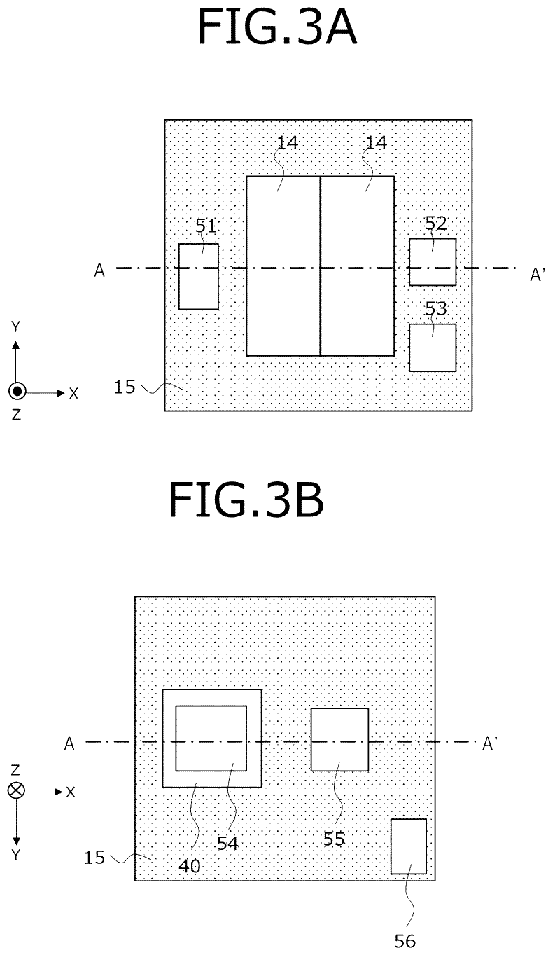

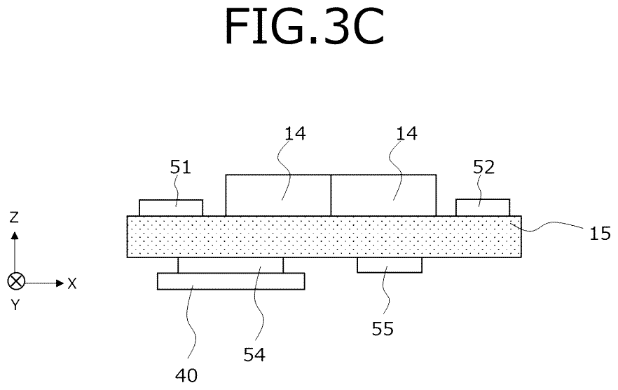

FIG. 3 is a schematic view showing an example of the periphery of the sensor substrate. FIG. 3A is a schematic view of the sensor substrate 15 as viewed from the lid 18 side (upper surface side), FIG. 3B is a schematic view of the sensor substrate 15 as viewed from the back side (lower surface side) of the surface facing the lid 18, and FIG. 3C is a schematic view corresponding to a cross-sectional view of the sensor substrate 15 along A-A′ in FIGS. 3A and 3B. In FIG. 3, a direction from a surface (lower surface) of the sensor substrate 15 on which the microcontroller 54 is provided to a surface (upper surface) of the sensor substrate 15 on which the sensor chip 14 and the sensor circuit are provided is defined as a Z-axis direction, and directions intersecting the Z-axis direction are defined as an X-axis direction and a Y-axis direction as shown in FIG. 3.

A sensor substrate 15 is provided inside the package 12, and a plurality of sensor chips 14 are arranged on the upper surface of the sensor substrate 15. At least one of the sensor chips 14 is a thermal conductivity (TC) sensor capable of detecting at least one selected from the group consisting of hydrogen, oxygen, and volatile organic compounds (VOCs). As the sensor chip 14 other than the TC sensor, at least one of a temperature sensor capable of measuring the temperature in the package 12 and a humidity sensor capable of measuring the humidity in the package 12 is disposed in the package 12. The sensor chip 14 is attached to a sensor substrate 15 and is disposed in the package 12. The distance from the upper surface of the sensor chip 14 to the lid 18 is preferably substantially constant. That is, it is preferable that the surface of the sensor chip 14 facing the lid 18 has a flat structure as much as possible. This is because the shape and arrangement are determined so that the gas flow in the first space is not blocked by the upper surface of the sensor chip 14. The sensor chip 14 and the sensor substrate 15 are preferably disposed at the center of the package 12.

Although not shown in the other drawings, as shown in FIG. 3A, a sensor circuit including an ADC (Analog-to-Digital Converter) circuit 51, a step-up converter circuit 52, and a step-down converter circuit 53 is provided on the upper surface of the sensor substrate 15 in addition to the sensor chip 14. The ADC circuit 51 is a circuit that converts the voltage and capacitance analog signals into voltage digital signals. The step-up converter circuit 52 and the step-down converter circuit 53 are used for adjusting the power supplied to the sensor chip 14. Other circuits may be provided in addition to these.

Although not shown in other drawings, as shown in FIG. 3B, the communication unit 40, the microcontroller 54, the power supply circuit 55, and the power supply unit 56 are provided on the lower surface of the sensor substrate 15. The communication unit 40 can transmit information on the detection result of the sensor chip 14 to an external device. The detection result includes, for example, information (data) on the concentration of the target detection object. The transmission may be performed, for example, by at least one of wired or wireless. The microcontroller 54 controls data communication, the sensor circuit, and the sensor chip 14. The microcontroller 54 includes a means for executing control based on a change in the detector in the sensor chip 14 as a software configuration that functions by executing a built-in control program. The power supply circuit 55 supplies an appropriate voltage to the microcontroller 54 and the sensor circuit. The power supply unit 56 is a portion connected to the battery 30 serving as a power source.

Although FIG. 3 shows an example in which the microcontroller 54 is provided on one surface of the sensor substrate 15 and the sensor circuit is provided on the other surface on the opposite side, a substrate on which the microcontroller 54 is provided and a substrate on which the sensor circuit is provided may be separately prepared. In the case described above, for example, by connecting the respective substrates with a cable connector or the like, a control signal from the microcontroller 54 is transmitted to the sensor circuit, and data acquired from the sensor circuit is transmitted to the microcontroller 54.

At least a part of the membrane 16 is, for example, a microporous membrane or a nonwoven fabric containing polytetrafluoroethylene (PTFE). The membrane 16 allows gas such as air, moisture, and gas to permeate therethrough, and thus the first space and the second space are not strictly isolated from each other. However, the membrane 16 does not allow liquid to permeate therethrough, and thus liquid cannot flow between the first space and the second space. The sensor chip 14 is disposed in the first space. In the present embodiment, the thickness and the pore size of the membrane 16 are not particularly limited, but when the detection device 1 is actually used, an appropriate membrane can be selected in accordance with the properties of the gas to be detected and the measurement environment.

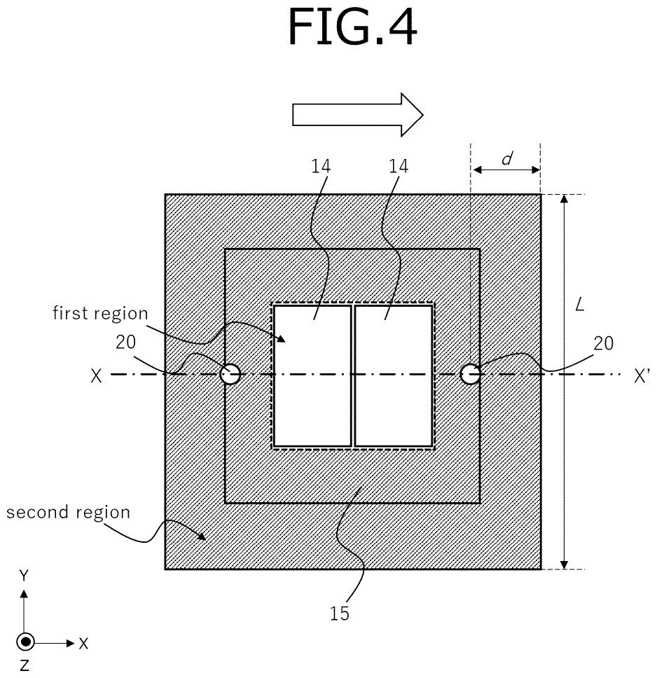

The lid 18 has a plurality of apertures 20. Since the gas flows in and out between the first space and the second space through the holes 20, the positions, sizes, shapes, and number of the holes 20 affect the flow of the gas in the first space. FIG. 4 is a schematic view showing an example of the lid 18 in the present embodiment. The lid 18 of the detection device 1 has two or more holes 20 in a second region which is a region outside a first region which is a region facing the sensor chip 14 in the lid 18. In FIG. 4, the second region is shown by hatching.

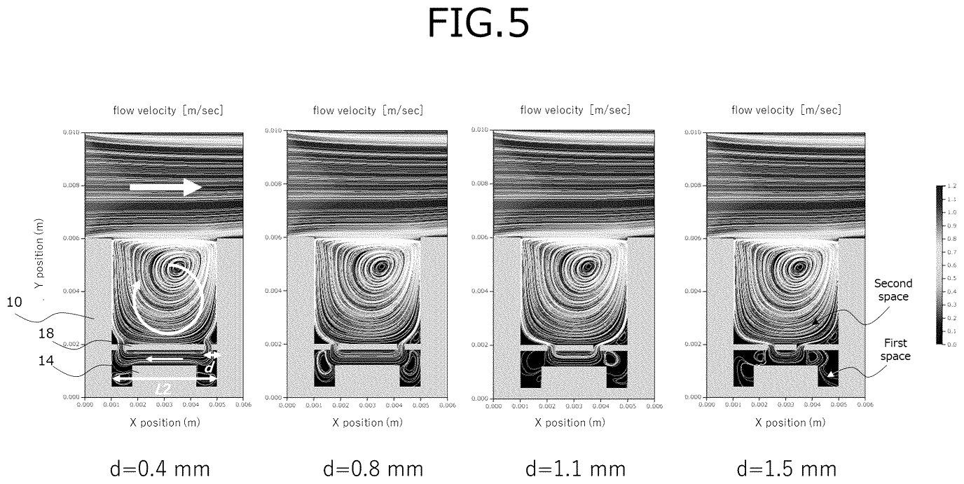

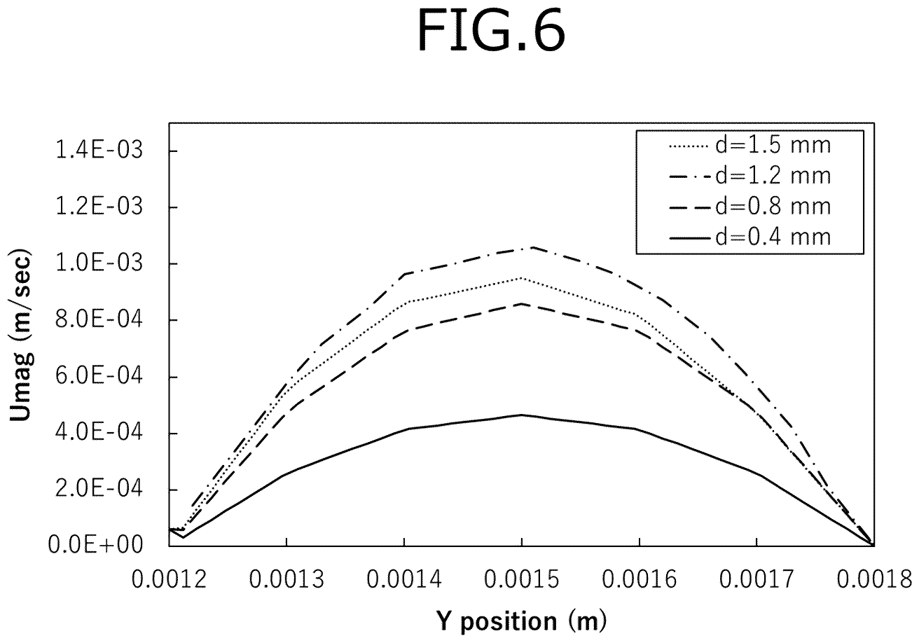

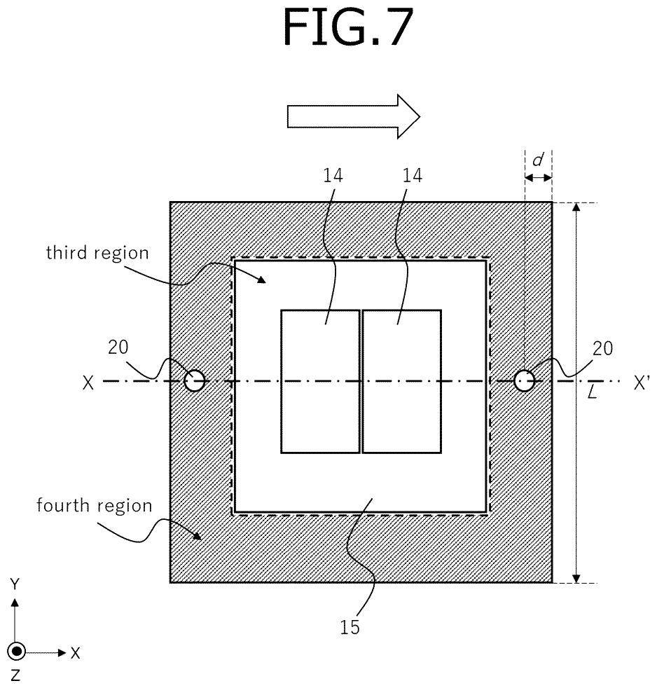

FIG. 5 shows a simulation result of the flow of the gas in the vicinity of the detection device 1. FIG. 5 is a sectional view taken along the chain line X-X′ in FIG. 4, showing the flow velocity. In the region on the opposite side of the first space with the second space interposed therebetween (hereinafter, described as “upper region of the detection device 1” in the present specification), there is a gas flow from the left side to the right side of the figure. This gas flow corresponds to the white arrows in FIG. 4. Under the influence of the gas flow, a vortex of the gas flow in the clockwise direction in the drawing is generated in the second space. Under the influence of the gas flow, an gas flow toward the left side of the figure is generated in the vicinity of the sensor chip 14 in the first space. The simulation was performed with the flow velocity of the gas flow toward the right side of the figure in the upper region of the detection device 1 being set to about 1 m/sec, and the flow velocity on the line connecting the center of the sensor chip 14 and the center of the lid 18 (the location where the value on the horizontal axis indicates 0.003 in FIG. 5) was calculated by the simulation. Here, the length between one of the inner walls of the package 12 and the other inner wall facing the one inner wall is denoted by L2, and the shortest length from the center of the hole 20 to the outer edge of a space (first space) surrounded by the package 12 and the lid 18 when the first space is vertically projected onto the lid 18 is denoted by d. The simulation was performed with the L2=4 mm. FIG. 5 shows the results when the ratio of the distances between the inner walls of the package 12 to the distances from the holes 20 to the inner walls is d/L2, and d/L2=0.1, 0.2, 0.3, and 0.4 in order from the left. The larger d/L2 means that the hole 20 is disposed closer to the inside of the lid 18. When d/L2=0.1 or d/L2=0.2, the hole 20 is present in the second region (the region outside the first region of the lid 18 facing the sensor chip 14) according to the setting conditions of the simulation. Since a uniform flow is formed between the two holes 20, the gas flow flowing on the upper surface of the sensor chip 14 can be made uniform when d/L2=0.1 or d/L2=0.2. On the other hand, when d/L2=0.3 or d/L2=0.4, in addition to the uniform flow from one side (right side in the figure) to the other side (left side in the figure) of the hole 20 in the first space, a flow is also generated outside the hole 20, and the flow on the upper surface of the sensor chip 14 is not uniform. FIG. 6 shows the results of a simulation of the velocity of the gas flow on the line connecting the center of the bottom surface of the first space and the center of the lid 18, with the position (value of d) of the hole 20 having a hole diameter of 0.3 mm being changed. FIG. 6 shows the results when d=0.4, 0.8, 1.2, and 1.5 mm. The values on the horizontal axis of the graph shown in FIG. 6 correspond to the vertical axis of FIG. 5. The position where the Y position shown on the horizontal axis is Y position=0.0012 [m] corresponds to the position of the upper surface of the sensor chip 14, and the position where the Y position is Y position=0.0018 [m] corresponds to the surface of the lid 18 in contact with the first space. Regardless of the position of the hole 20 (the value of d), the velocity of the gas flow in the center of the first space was the maximum value at a position (Y position=0.0015 [m]) where the distance from the upper surface of the sensor chip 14 and the distance from the surface of the lid 18 in contact with the first space were equal. The maximum value of the velocity was changed by changing the position of the hole 20 (the value of d). As d was increased to 0.4, 0.8, and 1.2 mm, the maximum value of the speed increased, and the maximum value in the case of d=1.5 mm was slightly smaller than that in the case of d=1.2 mm. That is, as long as the simulation is performed, it is found that the velocity of the gas flow in the center of the first space decreases as d decreases, and the uniformity of the velocity of the gas flow in the first space increases, with a peak at d=1.2 mm. When the measurement result of the TC sensor is corrected by the measurement result of the temperature sensor or the humidity sensor as in the detection device 1 according to the present embodiment, as a means for preventing the accuracy of correction from being reduced due to the difference in response speed between the sensors, it is conceivable to suppress the occurrence of turbulence in the first space, reduce the speed of the gas flow, and make the velocity of the gas flow uniform in the first space. Therefore, in the present embodiment, the position of the hole 20 provided in the lid 18 of the detection device 1 is preferably close to the package wall surface. In particular, the shortest length (d in this case) from the center of the hole 20 to the outer edge of the space (first space) surrounded by the package 12 and the lid 18 when the first space is vertically projected onto the lid 18 is ⅕ or less of the representative length (L2 in this case) of the first space. The lower limit of the ratio d/L2 is not particularly provided because it can vary depending on the diameter of the hole 20, but the hole 20 may be provided so as to contact the outer edge of the first space when the first space is vertically projected onto the lid 18. The ratio d/L2 is a ratio of the shortest distance d from the center of the hole 20 to the outer edge of the first space to the representative distance L2 of the first space. The representative lengths L2 of the first spaces are defined as the lengths of the long sides of the first spaces when the first spaces are rectangular, the diameters of the first spaces when the first spaces are circular, the lengths of the major axes of the first spaces when the first spaces are elliptical, and the maximum lengths of the shadows of the first spaces projected in a direction parallel to the lid 18 when the first spaces are other shapes. As shown in FIG. 4, the lid 18 has a hole 20 in a second region which is a region outside a first region which is a region facing the sensor chip 14. Further, as shown in FIG. 7, it is more preferable that two or more holes 20 are provided in a fourth region which is a region outside the third region which is a region facing the sensor substrate 15 in the lid 18. This is to make the gas flow flowing on the upper surface of the sensor chip 14 uniform. In FIG. 7, the fourth region is shown by hatching.

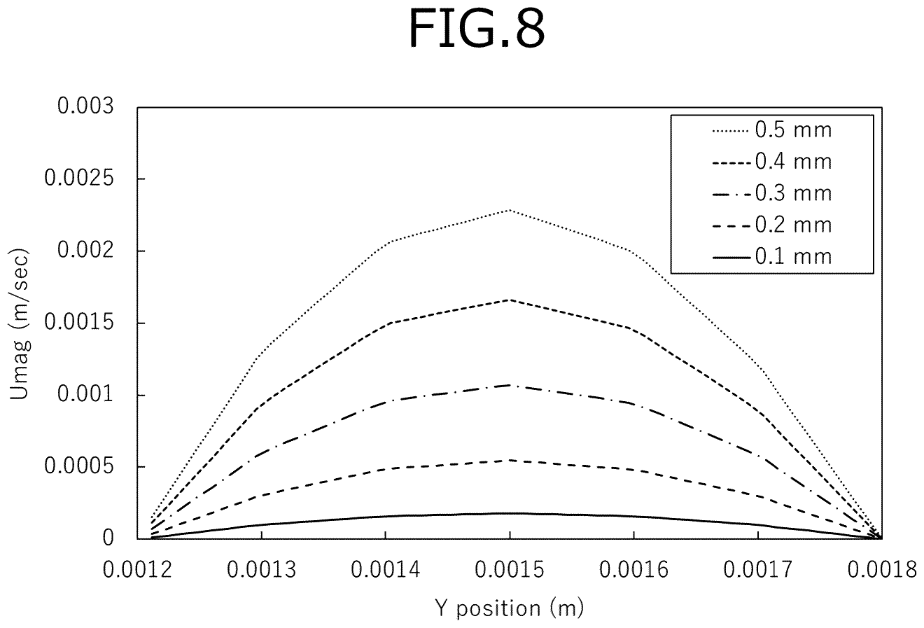

FIG. 8 shows the results of simulations of the velocity of the gas flow on the line connecting the center of the sensor chip 14 and the center of the lid 18 in the first space, with the hole diameter varied. The results are shown for the cases where the hole diameters at the positions of d=1.1 mm from the respective left and right package wall surfaces in the figure are 0.1, 0.2, 0.3, 0.4, and 0.5 mm. The values on the horizontal axis of the graph shown in FIG. 8 correspond to the vertical axis of FIG. 5. The position where the Y position shown on the horizontal axis is Y position=0.0012 [m] corresponds to the position of the upper surface of the sensor chip 14, and the position where the Y position is Y position=0.0018 [m] corresponds to the surface of the lid 18 in contact with the first space. The velocity of the gas flow in the center of the first space was the maximum value at a position (Y position=0.0015 [m]) where the distance from the upper surface of the sensor chip 14 and the distance from the surface of the lid 18 in contact with the first space were equal to each other, regardless of the diameter of each hole 20 from the package wall surface. Significant changes in the maximum value of the velocity were observed by varying the hole size. In the range of the hole diameter from 0.1 mm to 0.5 mm, the maximum value of the velocity decreased as the hole diameter decreased. That is, as long as the simulation is performed, it is found that the smaller the hole diameter is, the smaller the velocity of the gas flow at the center of the first space is, and the uniformity of the velocity of the gas flow in the first space is improved. As in the detection device 1 according to the present embodiment, when the measurement result of the TC sensor is corrected by the measurement result of the temperature sensor or the humidity sensor, as a means for preventing the accuracy of correction from being reduced due to the difference in response speed between the sensors, it is conceivable to reduce the speed of the gas flow in the first space and make the speed of the gas flow uniform. Therefore, the hole diameter of the hole 20 provided in the lid 18 of the detection device 1 is preferably sufficiently smaller than the dimension of the lid 18. Specifically, the hole diameter is preferably 0.5 mm or less, and more preferably 0.3 mm or less. The lower limit of the hole diameter is not particularly set, but it is considered that the hole diameter is preferably set to, for example, 0.05 mm or more because the gas to be measured needs to flow into the first space to some extent.

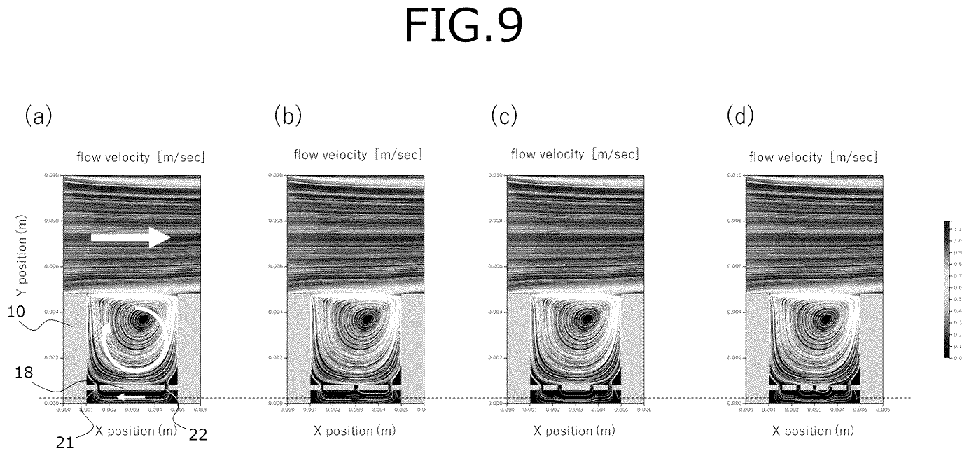

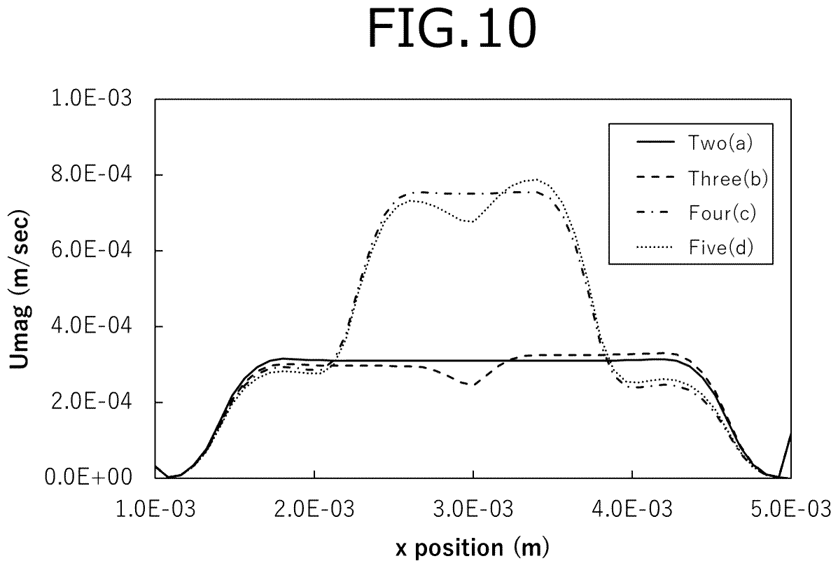

Next, in a case where one of the two holes 20 provided in the first region is a first hole 21 and the other hole 20 is a second hole 22, the number of holes along a line X-X′ connecting the first hole 21 and the second hole 22 will be described. In the lid 18 according to the present embodiment, the number of holes along X-X′ is preferably two in principle. That is, it is preferable that no hole is provided between the first hole 21 and the second hole 22 on X-X′. However, it does not mean that the problem is not solved and the effect of the invention is not exhibited as soon as another hole 20 is provided between two holes 20 along X-X′. For example, another hole 20 may be provided between two holes 20 along X-X′, and the number of holes 20 along X-X′ may be three. This is because the speed of the gas flow in the first space is uniform when the number of the holes 20 along X-X′ is two or three. When the number of the holes 20 along X-X′ is four or more, the speed of the gas flow in the first space becomes uneven, and therefore, the number of the holes 20 along X-X′ is preferably limited to two or three. FIG. 9 shows an example of a simulation result of the flow of gas in the vicinity of the detection device 1. FIG. 9 is a sectional view taken along line X-X′ in FIG. 4 or 7, showing the flow velocity. As in the simulation shown in FIG. 5, a gas flowing at about 1 m/sec toward the right side of the figure exists in the upper region of the detection device 1, and the L2 between one of the inner walls of the package 12 and the other inner wall facing the one inner wall is 4 mm. The hole diameter in FIG. 9 is 0.2 mm in all cases. In FIG. 9A, the number of the holes 20 along X-X′ is two, and the shortest distance between the center of each hole 20 and the outer edge of the lid 18 is 0.5 mm. The hole 20 on the left side in the figure is a first hole 21, and the hole 20 on the right side is a second hole 22. In FIG. 9B, the number of the holes 20 along X-X′ is three, and in addition to the case of FIG. 9A, another hole 20 is added between the first hole 21 and the second hole 22. In FIG. 9C, the number of the holes 20 along X-X′ is four, and in addition to the case of FIG. 9A, two holes 20 are added between the first hole 21 and the second hole 22, the shortest distance between the center of the hole 20 and the outer edge of the lid 18 being 1.3 mm. In FIG. 9D, the number of holes 20 along X-X′ is five, and the hole 20 at the center is added to FIG. 9C. FIG. 10 is a graph showing a velocity distribution of the gas flow measured along the horizontal axis of FIG. 9 at a position (Y position=0.0015 [m]) where the distance from the upper surface of the sensor chip 14 is equal to the distance from the surface of the lid 18 in contact with the first space. The results of FIGS. 9A, 9B, 9C and 9D are shown by a solid line, a dotted line, a broken line and a chain line, respectively. As shown in FIG. 10, the number of the holes 20 along X-X′ is roughly divided into two groups, that is, the case where the number of the holes 20 along X-X′ is two or three and the case where the number of the holes 20 along X-X′ is four or five. When the number of the holes 20 along X-X′ was four or five, the flow velocity near the center of the first space was more than twice the flow velocity when the number of the holes 20 along X-X′ was two or three. From this result, it is understood that the number of the holes 20 along X-X′ is preferably two or three in order to make the velocity distribution of the gas flow in the first space uniform. However, the number of holes 20 along X-X′ is not necessarily limited to two or three. The fast gas flow in the graphs of FIGS. 10C and 10D is considered to be caused by the influence of the holes 20 close to the center of the lid 18, and therefore, it is considered that the fast gas flow can be suppressed if the hole diameters of these holes are sufficiently smaller than the hole diameters of the outer holes. Therefore, for example, a small hole having a hole diameter equal to or smaller than ⅓ of the hole diameter of the outer hole 20 (the first hole 21 and the second hole 22) may be provided along X-X′ separately from two or three holes 20 along X-X′.

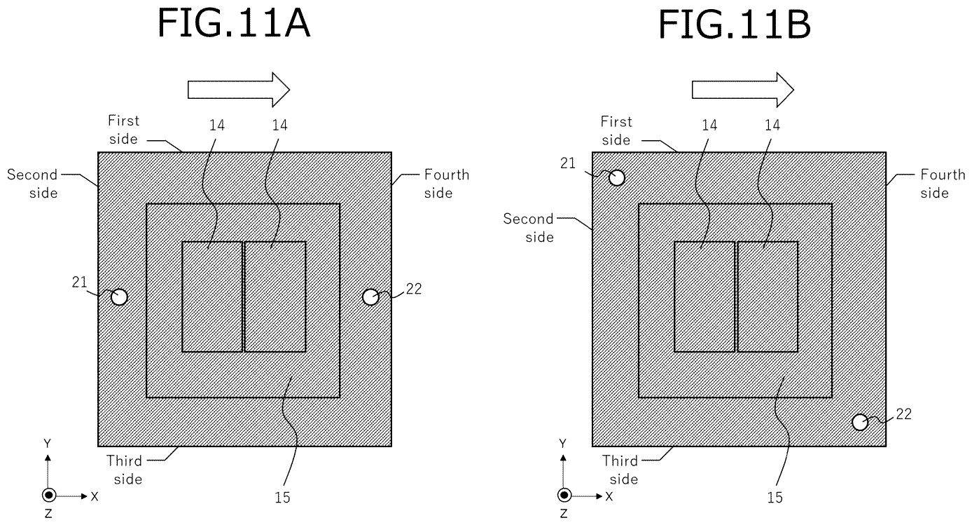

When the number of the holes 20 along X-X′ is two, if one of the holes 20 is a first hole 21 and the other is a second hole 22, the first hole 21 and the second hole 22 are preferably located at positions substantially symmetrical with respect to a point obtained by projecting the center point of the sensor chip 14 perpendicularly to the lid 18. Here, the center point of the sensor chip 14 is the center of gravity of a shadow figure projected when all the sensor chips 14 are vertically projected onto the lid 18 in the case where one or more sensor chips 14 exist. As shown in FIG. 11A, when one side of the outer periphery of the lid 18 is a first side, a side parallel to the first side is a third side, a side intersecting the first side is a second side, and a side parallel to the second side is a fourth side, the holes are preferably arranged such that the distance between the first hole 21 and the first side is equal to the distance between the first hole 21 and the third side, and the distance between the second hole 22 and the first side is equal to the distance between the second hole 22 and the third side. In FIG. 11, the white arrows indicate the direction of the gas flow in the upper region of the detection device 1. As shown in FIG. 11A, the straight line connecting the first hole 21 and the second hole 22 can be made substantially parallel to the gas flow indicated by the white arrow. In the present specification, the distance between the hole 20 and the side is described as the shortest distance between the center of the hole 20 and the outer edge of the lid 18.

However, the holes do not necessarily have to be arranged such that the distance between the first hole 21 and the first side is equal to the distance between the first hole 21 and the third side, and the distance between the second hole 22 and the first side is equal to the distance between the second hole 22 and the third side. That is, the straight line connecting the first hole 21 and the second hole 22 is not necessarily parallel to the gas flow in the upper region of the detection device 1. As shown in FIG. 11B, the straight line connecting the two holes may intersect the gas flow. In the example shown in FIG. 11B, the holes 20 are arranged in regions near the corners of the lid 18. When one side of the outer periphery of the lid 18 is a first side, a side parallel to the first side is a third side, a side intersecting the first side is a second side, and a side parallel to the second side is a fourth side, the distance between the first hole 21 and the first side is equal to the distance between the first hole 21 and the second side, and the distance between the second hole 22 and the third side is equal to the distance between the second hole 22 and the fourth side.

By providing the hole 20 having a diameter equal to or smaller than a predetermined size in the outer portion of the lid 18, specifically, in the second region or the like, the flow velocity in the package can be reduced, and the time constant associated with the gas inflow into the package 12 can be made larger than the time constant of the sensor chip 14 having the largest time constant. This makes it possible to achieve accurate correction in the transient response of the detection device.

The detection device 1 may further include a communication unit. The communication unit may be disposed inside the first space or outside the first space. The communication unit can transmit information on the detection result of the sensor chip 14 to an external device. The detection result includes, for example, information (data) on the concentration of the target detection object. The transmission may be performed, for example, by either wired or wireless.

### (First Modification)

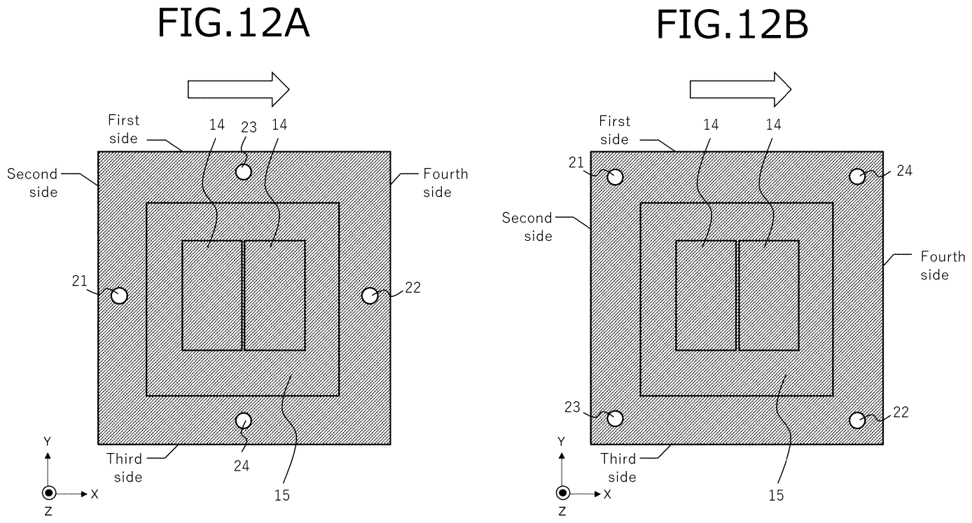

According to the present modification, the lid 18 of the detection device 1 may have the hole 20 in addition to the first hole 21 and the second hole 22. For example, according to the aspect shown in FIG. 12, the lid 18 of the detection device 1 further includes a third hole 23 and a fourth hole 24 in addition to the first hole 21 and the second hole 22, the distance between the third hole 23 and the first hole 21 is equal to the distance between the third hole 23 and the second hole 22, and the distance between the fourth hole 24 and the first hole 21 is equal to the distance between the fourth hole 24 and the second hole 22. As shown in FIG. 12A, when one side of the outer periphery of the lid 18 is a first side, a side parallel to the first side is a third side, a side intersecting the first side is a second side, and a side parallel to the second side is a fourth side, the holes 20 are arranged such that the distance between the first hole 21 and the first side is equal to the distance between the first hole 21 and the third side, the distance between the second hole 22 and the first side is equal to the distance between the second hole 22 and the third side, the distance between the third hole 23 and the second side is equal to the distance between the third hole 23 and the fourth side, and the distance between the fourth hole 24 and the second side is equal to the distance between the fourth hole 24 and the fourth side. In FIG. 12B, the holes 20 are arranged in the region near the corner of the lid 18, and the distance between the first hole 21 and the first side is equal to the distance between the first hole 21 and the second side, the distance between the second hole 22 and the third side is equal to the distance between the second hole 22 and the fourth side, the distance between the third hole 23 and the second side is equal to the distance between the third hole 23 and the third side, and the distance between the fourth hole 24 and the first side is equal to the distance between the fourth hole 24 and the fourth side.

### (Second Modification)

According to the present modification, the shape of the hole 20 is not limited to a circular shape. The shape may be an ellipse, a square, a rectangle, a polygon or the like. In this case, when the maximum representative length such as the major axis of an ellipse or the dimension of the longer side of a square or rectangle is defined as the hole size, the hole size is preferably 0.5 mm or less, and more preferably 0.3 mm or less.

### (Third Modification)

According to the present modification, the shape of the package 12 as viewed from the direction of the lid 18 is not limited to a square. The shape may be circular, elliptical, rectangular, polygonal or the like.

When the package 12 has a circular shape as viewed from the lid 18, the distance between the hole 20 and the outer edge of the lid 18 is preferably ⅕ or less of the diameter of the package 12. When the package 12 has an elliptical shape as viewed from the lid 18, the distance between the hole 20 and the outer edge of the lid 18 is preferably ⅕ or less of the major axis. When the package 12 has a rectangular shape as viewed from the direction of the lid 18, the distance between the hole 20 and the outer edge of the lid 18 is preferably ⅕ or less of the long side. If the shape of the package 12 viewed from the direction of the lid 18 is polygonal shape, the dimension parallel to the direction in which the distance between the hole 20 and the outer edge is measured is defined as the representative length, and the length is preferably ⅕ or less of the representative length. When the package 12 is rectangular as viewed from the lid 18, and the lid 18 is rectangular having a first long side, a second long side parallel to the first long side, a first short side shorter than the first long side, and a second short side parallel to the first short side, the distance between the first hole and the first long side is preferably equal to the distance between the first hole and the second long side, and the distance between the second hole and the first long side is preferably equal to the distance between the second hole and the second long side. In the same case, it is preferable that the distance between the first hole and the first short side is equal to the distance between the first hole and the second short side, and the distance between the second hole and the first short side is equal to the distance between the second hole and the second short side.

### Second Embodiment

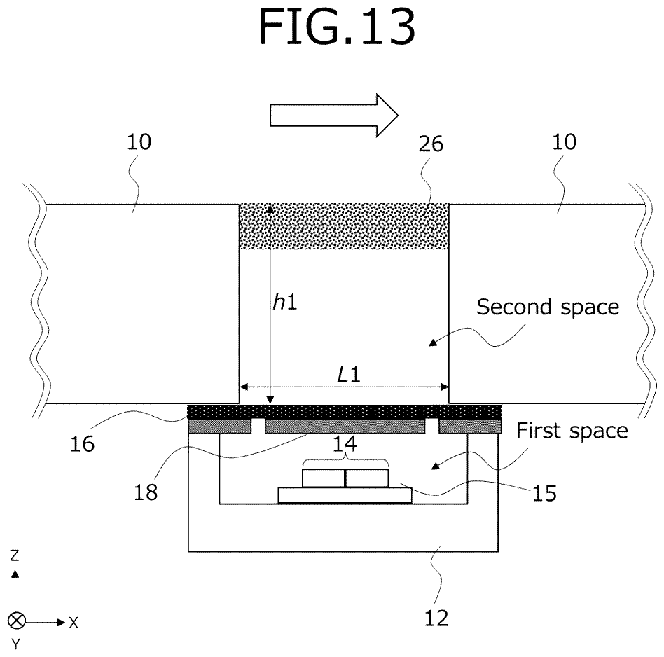

FIG. 13 is a schematic sectional view showing a part of the detection device 1 according to the present embodiment. In the present embodiment, an gas flow decelerating member 26 such as a filter is provided in an opening between the second space surrounded by the housing wall 10 intersecting the lid 18 and the upper region of the detection device 1. The gas flow decelerating member 26 is a physical means for decelerating the gas flow in the second space. The gas flow decelerating member 26 is a filter made of, for example, PTFE which is a porous material having a pore diameter of 0.1 μm or more and 1.0 μm or less. In addition, the gas flow decelerating member 26 may be a porous body containing zeolite, silica, or an organic material, a metal mesh, a nonwoven fabric, a fiber body, a brush, or the like, and the material of the gas flow decelerating member is not limited.

The arrangement of the gas flow decelerating member 26 is not limited to the opening in the figure. The gas flow decelerating member 26 may be disposed at any position as long as the gas flow decelerating member 26 can decelerate the gas flow flowing in the first space and the second space. For example, the gas flow decelerating member 26 may be disposed at the bottom of the second space, between the bottom and the opening of the second space, or outside the second space (near the white arrow in the drawing). The gas flow decelerating member 26 may be disposed in the first space, for example, between the sensor chip 14 and the lid 18.

### Example 1

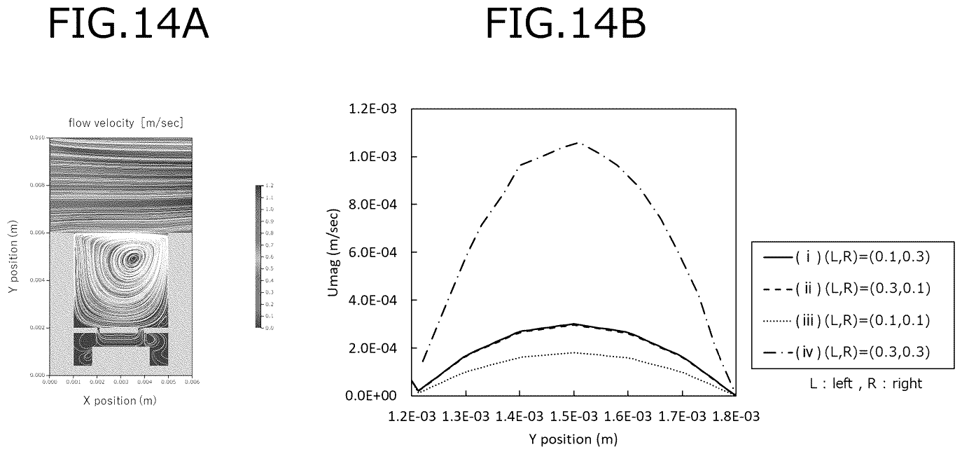

FIG. 14 shows the results of simulation in which the hole diameter of each of the holes 20 is changed when there are two holes 20 along X-X′. As shown in FIG. 14A, when the distance from X position=1 mm to 5 mm on X-X′ corresponds to the length of the second space, the two holes 20 were arranged at X position=2.1 mm and 3.9 mm, respectively, that is, d=1.1 mm. When the numerical values of the X positions of the holes 20 are arranged in ascending order (left, right), the simulated hole diameters (mm) have the following four patterns.

$$( left , right ) = ( 0.1 , 0.3 ) \qquad ( i )$$

$$( left , right ) = ( 0.3 , 0.1 ) \qquad ( ii )$$

$$( left , right ) = ( 0.1 , 0.1 ) \qquad ( iii )$$

$$( left , right ) = ( 0.3 , 0.3 ) \qquad ( iv )$$

The simulation was performed with the flow velocity of the gas flow toward the right side of the drawing in the upper region of the detection device 1 being set to about 1 m/sec.

FIG. 14B is a graph showing the distribution of the flow velocity in the Y direction at X position=3 mm, where the direction of Y position in the figure is the Y direction. The simulation results of (i) are shown by a solid line, (ii) by a broken line, (iii) by a dotted line, and (iv) by a chain line. It is understood from FIG. 14B that the flow velocity is particularly high in the case of (iv) (left, right)=(0.3, 0.3) among the four patterns. On the other hand, in the case of (i) (left, right)=(0.1, 0.3) or (ii) (left, right)=(0.3, 0.1), the flow velocity is significantly lower than in the case of (iv), and it has been found that the flow velocity is limited by the smaller hole diameter. Since the graphs showing (i) and (ii) overlap in FIG. 14B, it is understood that there is no influence even if the size relationship of the hole diameters is reversed if the combination of the hole diameters is the same.

### Example 2

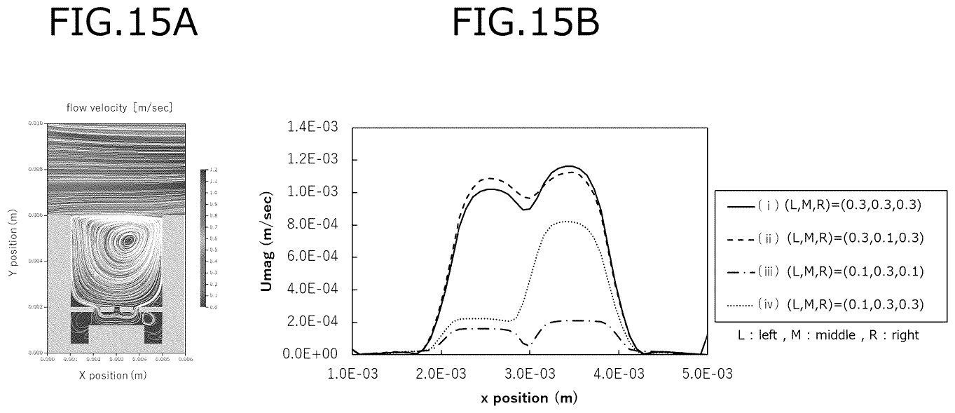

FIG. 15 shows the results of simulation in which the hole diameter of each hole 20 is changed when there are three holes 20 along X-X′. As shown in FIG. 15A, when the distance from X position=1 mm to 5 mm on X-X′ corresponds to the length of the second space, the three holes 20 were arranged at positions of X position=2.2 mm, 3.0 mm, and 3.8 mm, respectively. When the numerical values of the X positions of the holes 20 are arranged in ascending order (left, middle, and right), the simulated hole diameters (mm) have the following four patterns.

$$( left , middle , right ) = ( 0.3 , 0.3 , 0.3 ) \qquad ( i )$$

$$( left , middle , right ) = ( 0.3 , 0.1 , 0.3 ) \qquad ( ii )$$

$$( left , middle , right ) = ( 0.1 , 0.3 , 0.1 ) \qquad ( iii )$$

$$( left , middle , right ) = ( 0.1 , 0.3 , 0.3 ) \qquad ( iv )$$

The simulation was performed with the flow velocity of the gas flow toward the right side of the figure in the upper region of the detection device 1 being set to about 1 m/sec.

FIG. 15B is a graph showing the distribution of the flow velocity along the X direction. The simulation result of (i) is shown by a solid line, (ii) by a broken line, (iii) by a dotted line, and (iv) by a chain line. In the graphs of (i), (iii), and (iv), there is a difference between the right and left sides of X position=3.0 mm, and the flow velocity on the right side corresponding to the downstream is larger than that on the left side, whereas in the graph of (ii), there is not so remarkable difference between the right and left sides as in other cases. From this result, it is found that, when three holes are provided in a straight line, the size of the hole in the middle is smaller than the holes at both ends, and thus the distribution of the gas flow becomes uniform. Therefore, for example, when another hole is provided between the first hole 21 and the second hole 22 on the line connecting the first hole 21 and the second hole 22, and these three holes are provided in a straight line, the size of the hole between the first hole 21 and the second hole 22 is preferably equal to or smaller than the size of the smaller hole of the first hole 21 and the second hole 22.

### Example 3

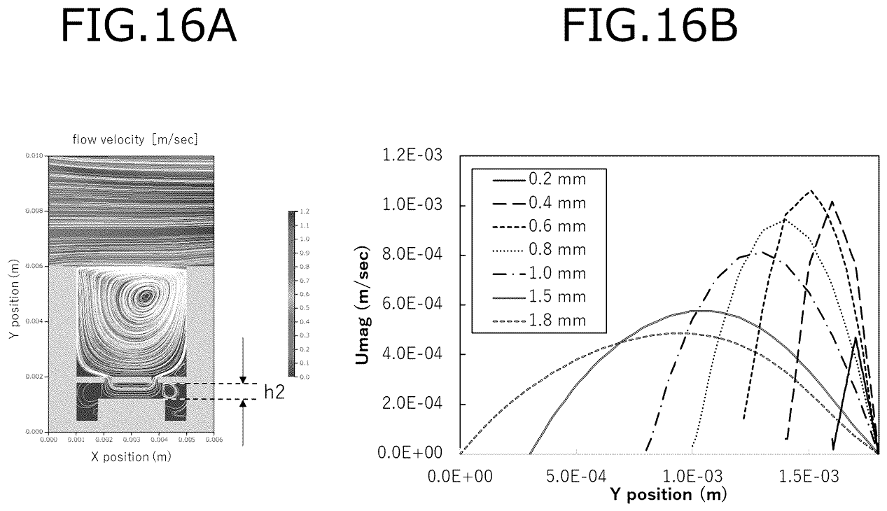

FIG. 16 shows the results of simulation performed by changing the distances h2 between the lid 18 and the sensors included in the sensor chip 14 when there are two holes 20 along X-X′. As shown in FIG. 16A, when the distance from X position=1 mm to 5 mm on X-X′ corresponds to the length of the second space, the two holes 20 were arranged at positions of X position=2.1 mm and 3.9 mm, respectively. When the hole diameters of the two holes 20 are 0.3 mm, the simulation was performed with seven patterns of distances h2 between the back surface of the lid 18 and the upper surface of the sensor chip 14, i.e., (i) 0.2 mm, (ii) 0.4 mm, (iii) 0.6 mm, (iv) 0.8 mm, (v) 1.0 mm, (vi) 1.5 mm, and (vii) 1.8 mm. The simulation was performed with the flow velocity of the gas flow toward the right side of the drawing in the upper region of the detection device 1 being set to about 1 m/sec.

FIG. 16B is a graph showing the distribution of the flow velocity in the Y direction when the X position is 3 mm. The simulation result of (i) is shown by a solid line, (ii) by a thin broken line, (iii) by a coarse broken line, (iv) by a dotted line, (v) by a chain line, (vi) by a white solid line, and (vii) by a white broken line. According to FIG. 16B, in the seven patterns, under the condition of h2=0.6 mm or less, the maximum value of the flow velocity increases as the h2 increases, but the maximum value of the flow velocity decreases as the h2 increases, with h2=0.6 mm as a boundary. However, even if the h2 condition is changed within the range, the distribution of the flow velocity is substantially symmetrical between the left and right along the X-axis. Here, it is preferable that the flow in the first space is uniform and the difference between the left and right sides is small. As for the type of the flow generated in the first space, in any of the cases (i) to (vii), no turbulent flow was generated as in the model shown in FIG. 16A. Therefore, in this embodiment, h2 may be any value from 0.2 mm to 1.8 mm.

However, depending on the arrangement position, the number, the shape, and the like of the holes 20, even when the h2 is set to be 0.2 mm or more and 1.8 mm or less, turbulence may occur in the first space. In this case, the detection accuracy of the detection device 1 can be improved by appropriately adjusting the value of the h2 so that turbulence does not occur in the first space.

### Example 4

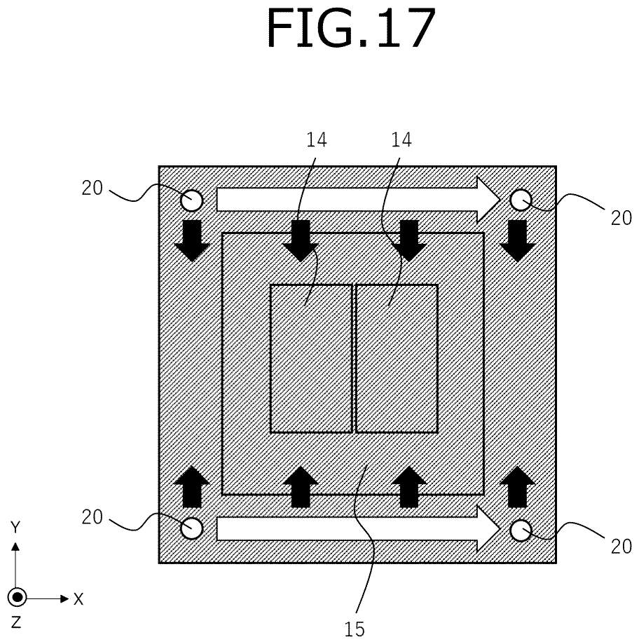

In the above-described embodiment and the like, the flow velocity distribution in the first space is examined along the flow velocity direction of the gas flow, but in the present embodiment, the gas flow in the first space in the direction substantially perpendicularly intersecting the flow velocity direction of the gas flow is examined. FIG. 17 is a schematic view showing the relationship between the gas flow and the diffusion direction. The gas flowing in the upper region of the detection device 1 flows from the left side to the right side in the figure. The direction of the gas flow in the first space along the gas flow is indicated by white arrows, and the state where the gas flow flowing in from the hole 20 diffuses in the direction substantially perpendicular to the white arrows is indicated by black arrows. In this embodiment, it is assumed that the movement of the gas in the direction of the black arrow is caused by diffusion. Diffusion in the direction of the black arrow is considered, with the concentration along the white arrow being constant and the surface concentration being constant. As for the diffusion amount, the normalized diffusion amount of hydrogen with respect to time was calculated from Fick's law.

The normalized diffusion amount N(z,t)/N₀ at a distance z [mm] from the diffusion source t seconds after the start of diffusion can be calculated by the following mathematical formula (1).

$$\frac{N ( z , t )}{{N}_{0}} = erfc [ \frac{z}{2 \sqrt{Dt}} ] \qquad [ Formula 1 ]$$

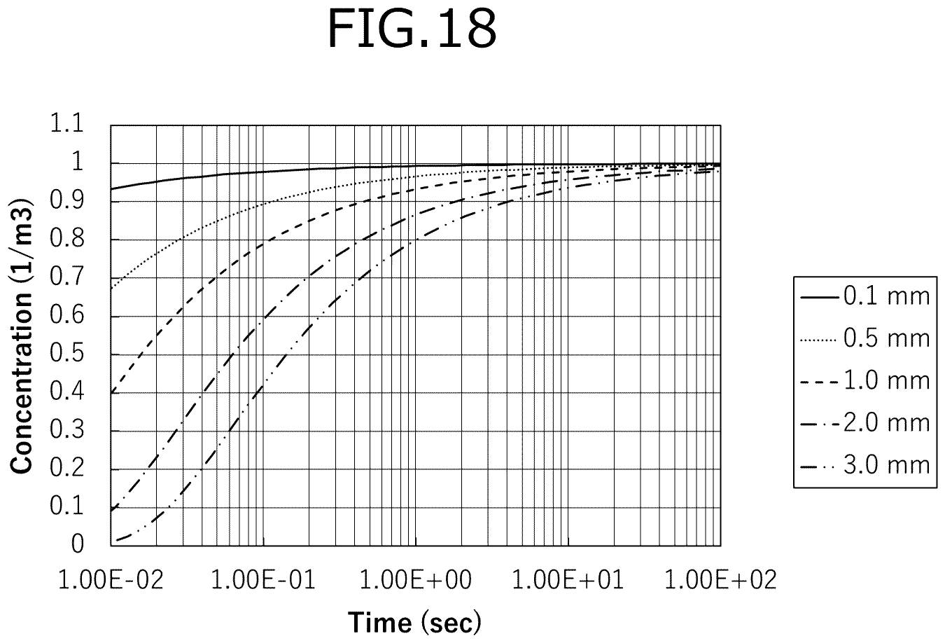

FIG. 18 is a graph showing a change over time in the normalized diffusion amount N(z,t)/N₀ calculated by formula (1). The simulation result of (i) z=0.1 mm is shown by a solid line, the simulation result of (ii) z=0.5 mm is shown by a dotted line, the simulation result of (iii) z=1.0 mm is shown by a broken line, the simulation result of (iv) z=2.0 mm is shown by a one dot chain line, and the simulation result of (v) z=3.0 mm is shown by a two dot chain line. It is understood from FIG. 18 that, although diffusion takes a longer time as z increases, about 85% of the response is obtained after 1.0 seconds even in the case of z=2.0 mm. For example, since the dimensions of the inside of the package 12 used in the simulation in the present specification are the vertical and horizontal 4 mm, it can be considered that 85% or more of the gas reaches the center of the package 12 after 1.0 seconds have elapsed, and sufficient gas movement occurs in a short time by diffusion even in a direction in which there is no flow. Therefore, according to the present embodiment, it is considered that diffusion in the direction perpendicular to the flow velocity also occurs at a sufficient speed, and the gas flow in the first space becomes uniform without any problem.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. These embodiments and modifications thereof are included in the scope and spirit of the invention, and are also included in the invention described in the claims and the scope of equivalents thereof.

The invention of the embodiment will be described below.

<1>

A detection device including:
a package having a lid, and
a sensor substrate located inside the package and holding a sensor chip, wherein
the lid has two or more holes in a second region that is a region outside a first region that is a region of the lid facing the sensor chip.
   <2>

The detection device according to <1>, wherein
the lid has the hole in a fourth region that is a region outside a third region that is a region of the lid facing the sensor substrate.
   <3>

A detection device including:
a package having a lid, and
a sensor substrate located inside the package and holding a sensor chip, wherein
the lid has two or more holes, and a shortest distance from a center of the hole to an outer edge of a space surrounded by the package and the lid when the space is vertically projected on the lid is equal to or less than one fifth of a representative length of the space.
   <4>

The detection device according to any one of <1> to <3>, wherein
a first hole and a second hole are located at positions substantially symmetrical with respect to a point obtained by projecting a center point of the sensor chip perpendicularly to the lid.
   <5>

The detection device according to any one of <1> to <4>, wherein
the lid has a first side, a third side parallel to the first side, a second side intersecting the first side, and a fourth side parallel to the second side,
a distance between the first hole and the first side is equal to a distance between the first hole and the third side, and
a distance between the second hole and the first side is equal to a distance between the second hole and the third side.
   <6>

The detection device according to any one of <1> to <5>, wherein
the lid has a first side, a third side parallel to the first side, a second side intersecting the first side, and a fourth side parallel to the second side,
a distance between the first hole and the first side is equal to a distance between the first hole and the second side, and
a distance between the second hole and the third side is equal to a distance between the second hole and the fourth side.
   <7>

The detection device according to any one of <1> to <6>, further including,
a third hole that is one of the holes and
a fourth hole that is one of the holes, wherein
a distance between the third hole and the first hole is equal to a distance between the third hole and the second hole, and
a distance between the fourth hole and the first hole is equal to a distance between the fourth hole and the second hole.
   <8>

The detection device according to any one of <1> to <7>, wherein
a distance between the third hole and the second side is equal to a distance between the third hole and the third side, and
a distance between the fourth hole and the first side is equal to a distance between the fourth hole and the fourth side.
   <9>

The detection device according to any one of <1> to <8>, wherein
the lid does not have a hole between the first hole and the second hole on a line connecting the first hole and the second hole.
   <10>

The detection device according to any one of <1> to <8>, wherein
the lid has one hole between the first hole and the second hole on a line connecting the first hole and the second hole, and
a hole diameter of the hole located between the first hole and the second hole on a line connecting the first hole and the second hole is equal to or smaller than a smaller one of the hole diameters of the first hole and the second hole.
   <11>

The detection device according to any one of <1> to <10>, further including,
an gas flow decelerating member including any one of a filter, a porous body, a metal mesh, a nonwoven fabric, a fiber body, and a brush,
the lid is provided between the gas flow decelerating member and the sensor chip, or the gas flow decelerating member is provided between the lid and the sensor chip.
   <12>

The detection device according to any one of <1> to <11>, wherein
the hole diameter of the hole is 0.05 mm or more and 0.5 mm or less.

What is claimed is:

1. A detection device comprising:
a package having a lid, and
a sensor substrate located inside the package and holding a sensor chip, wherein
the lid has two or more holes in a second region that is a region outside a first region that is a region of the lid facing the sensor chip.

2. The detection device according to claim 1, wherein
the lid has the hole in a fourth region that is a region outside a third region that is a region of the lid facing the sensor substrate.

3. A detection device comprising:
a package having a lid, and
a sensor substrate located inside the package and holding a sensor chip, wherein
the lid has two or more holes, and a shortest distance from a center of the hole to an outer edge of a space surrounded by the package and the lid when the space is vertically projected on the lid is equal to or less than one fifth of a representative length of the space.

4. The detection device according to claim 1, wherein
a first hole and a second hole are located at positions substantially symmetrical with respect to a point obtained by projecting a center point of the sensor chip perpendicularly to the lid.

5. The detection device according to claim 3, wherein
a first hole and a second hole are located at positions substantially symmetrical with respect to a point obtained by projecting a center point of the sensor chip perpendicularly to the lid.

6. The detection device according to claim 4, wherein
the lid has a first side, a third side parallel to the first side, a second side intersecting the first side, and a fourth side parallel to the second side,
a distance between the first hole and the first side is equal to a distance between the first hole and the third side, and
a distance between the second hole and the first side is equal to a distance between the second hole and the third side.

7. The detection device according to claim 4, wherein
the lid has a first side, a third side parallel to the first side, a second side intersecting the first side, and a fourth side parallel to the second side,
a distance between the first hole and the first side is equal to a distance between the first hole and the second side, and
a distance between the second hole and the third side is equal to a distance between the second hole and the fourth side.

8. The detection device according to claim 4 further comprising
a third hole that is one of the holes and
a fourth hole that is one of the holes, wherein
a distance between the third hole and the first hole is equal to a distance between the third hole and the second hole, and
a distance between the fourth hole and the first hole is equal to a distance between the fourth hole and the second hole.

9. The detection device according to claim 7, wherein
a distance between the third hole and the second side is equal to a distance between the third hole and the third side, and
a distance between the fourth hole and the first side is equal to a distance between the fourth hole and the fourth side.

10. The detection device according to claim 4, wherein
the lid does not have a hole between the first hole and the second hole on a line connecting the first hole and the second hole.

11. The detection device according to claim 4 wherein
the lid has one hole between the first hole and the second hole on a line connecting the first hole and the second hole, and
a hole diameter of the hole located between the first hole and the second hole on a line connecting the first hole and the second hole is equal to or smaller than a smaller one of the hole diameters of the first hole and the second hole.

12. The detection device according to claim 1, further comprising:
a gas flow decelerating member including any one of a filter, a porous body, a metal mesh, a nonwoven fabric, a fiber body, and a brush,
the lid is provided between the gas flow decelerating member and the sensor chip, or the gas flow decelerating member is provided between the lid and the sensor chip.

13. The detection device according to claim 1, wherein
the hole diameter of the hole is 0.05 mm or more and 0.5 mm or less.

14. The detection device according to claim 3, wherein
the hole diameter of the hole is 0.05 mm or more and 0.5 mm or less.
